(19) United States
(12) Patent Application Publication
(10) Pub. No.: US 2023/0136792 A1
(43) Pub. Date: May 4, 2023

(54) COMPOSITIONS AND METHODS FOR TREATING AGE-RELATED DISEASES AND PREMATURE AGING DISORDERS

(71) Applicant: Sirtsei Pharmaceuticals, Inc., Morrisville, NC (US)

(72) Inventors: Michael Friedrich Ackermann, Holly Springs, NC (US); Kelly J. Abernathy, Cary, NC (US); Gregory Cooksey Rigdon, Wake Forest, NC (US); Stephen E. Butts, Apex, NC (US)

(21) Appl. No.: 17/907,732

(22) PCT Filed: Apr. 1, 2021
(86) PCT No.: PCT/US2021/025295

§ 371 Date: Sep. 29, 2022

Related U.S. Application Data
(60) Provisional application No. 63/003,977, filed on Apr. 2, 2020.

(51) Int. Cl. A61K 31/4245; A61K 31/454; A61K 31/422; A61K 31/424; A61K 31/506; A61K 31/4439

(52) U.S. Cl. CPC A61K 31/4245; A61K 31/454; A61K 31/422; A61K 31/424; A61K 31/506; A61K 31/4439

(57) ABSTRACT

The present invention relates to methods of treating, delaying the onset of, slowing the progression of, or reducing the severity of age-related diseases by administering a compound of Formula 1 to a subject in need thereof. The invention further relates to methods of treating, delaying the onset of, slowing the progression of, or reducing the severity of premature aging disorders by administering a compound of Formula 1 to a subject in need thereof.

### STATEMENT OF PRIORITY

This application claims the benefit of U.S. Provisional Application Ser. No. 63/003,977, filed Apr. 2, 2020, the entire contents of which are incorporated by reference herein.

### FIELD OF THE INVENTION

The present invention relates to methods of treating, delaying the onset of, slowing the progression of, or reducing the severity of age-related diseases by administering a compound of Formula 1 to a subject in need thereof. The invention further relates to methods of treating, delaying the onset of, slowing the progression of, or reducing the severity of premature aging disorders by administering a compound of Formula 1 to a subject in need thereof.

### BACKGROUND OF THE INVENTION

Progeroid syndromes (PS) are a group of rare genetic disorders which mimic physiological aging, making affected individuals appear to be older than they are. All disorders within this group are thought to be monogenic. Most known PS are due to genetic mutations that lead to either defects in the DNA repair mechanism (e.g., RecQ protein-like helicases (RECQLs), nucleotide excision repair (NER) proteins) or defects in nuclear envelope proteins (e.g., lamin A/C).

Examples of PS include Hutchinson—Gilford progeria syndrome, Werner syndrome, Bloom syndrome, Rothmund—Thomson syndrome, Cockayne syndrome, xeroderma pigmentosum, trichothiodystrophy, combined xeroderma pigmentosum-Cockayne syndrome, and restrictive dermopathy.

Hutchinson—Gilford progeria syndrome (HGPS) is one of the most widely studied PS disorders as it most resembles natural ageing. HGPS is a rare, fatal, generic condition of childhood with features resembling premature aging. After what appears a normal birth, affected children begin to experience growth delays resulting in short stature and low weight and develop a disproportionately small face in comparison to the head with distinct facial features resembling that of old people.

The syndrome is also associated with other diseases prominent in senior citizens such as generalized atherosclerosis, cardiovascular disease and stroke, hip dislocations, unusually prominent veins of the scalp, loss of the layer of fat beneath the skin (subcutaneous adipose tissue), defects of the nails, joint stiffness, skeletal defects among others and shortened telomeres. According to reports in the medical literature, individuals with HGPS develop premature, widespread thickening and loss of elasticity of artery walls (arteriosclerosis), which result in life-threatening complications during childhood, adolescence, or early adulthood. Children with progeria die of heart disease (atherosclerosis) at an average age of 13 years, with a range of about eight to 21 years. As with any person suffering from heart disease, the common events as heart disease advances for children with progeria can include high blood pressure, strokes, angina (chest pain due to poor blood flow to the heart itself), enlarged heart, and heart failure, all conditions associated with aging.

[HGPS is a rare disorder that appears to affect males and females and races equally. The disorder was originally described in the medical literature in 1886 (J. Hutchinson) and 1897 (H. Gilford). As of January 2014, approximately 200 cases have been reported. Estimates indicate that the prevalence of HGPS is approximately one in eighteen million, thus at any given time, there are approximately 350-400 children living with progeria worldwide.

The cause of HGPS is a single-letter mutation in a gene on chromosome 1 that codes for lamin A (C→T transition at nucleotide 1824 (G608G)), activating a splice donor site that results in production of a dominant negative form of lamin A protein, denoted progerin. The lamin A protein is a key component of the nuclear membrane and provides the scaffolding that holds the nucleus of a cell together. The developing cellular instability when the mutation is present appears to lead to the process of premature aging in progeria.

Normally Lamin A undergoes several processing steps including C-terminal farnesylation and subsequent cleavage by the endoprotease Zmpste24 (FACE-1) as well as a tail methylation by isoprenyl cysteine carboxylmethyltransferase. Progerin fails to undergo the complete processing because it lacks the Zmpste24 cleavage site as a consequence of aberrant splicing in LMNA exon 11 caused by the disease mutation.

A similar splicing event occurs in vivo at a low level in the skin at all ages. Progerin-positive cells are present in primary fibroblast cultures obtained from the skin of normal donors at advanced ages. These cells display HGPS-like defects in nuclear morphology, decreased H3K9me3 and HP1, and increased histone H2AX phosphorylation marks of the DNA damage loci. Inhibition of progerin production in cells of aged non-HGPS donors in vivo increases the proliferative activity, H3K9me3, and HP1, and decreases the senescence markers p21, IGFBP3, and GADD45B to the levels of young donor cells. Thus, progerin-dependent mechanisms act in natural aging. Excessive activity of the same mechanisms may well be the cause of premature aging in HGPS. Telomere attrition is widely regarded to be one of the primary hallmarks of aging. Progerin expression in normal human fibroblasts accelerates the loss of telomeres. Changes in lamina organization may directly affect telomere attrition resulting in accelerated replicative senescence and progeroid phenotypes. The chronological aging in normal individuals and the premature aging in HGPS patients are mediated by similar changes in the activity of signaling pathways, including downregulation of DNA repair and chromatin organization, and upregulation of ERK, mTOR, GH-IGF1, MAPK, TGFβ, and mitochondrial dysfunction (Ashapkin et al., Front. Genet. 15 May 2019).

One of the functions of Lamin A is activation of sirtuin 6 (SIRT6), a stress responsive protein deacetylase and mono-ADP ribosyltransferase enzyme encoded by the SIRT6 gene. SIRT6 functions in multiple molecular pathways related to aging, including DNA repair, telomere maintenance, glycolysis and inflammation. SIRT6 activation facilitates chromatin localization of SIRT6 upon DNA damage. Lamin A promotes SIRT6-dependent DNA-PKcs (DNA-PK catalytic subunit recruitment to chromatin, CtlP deacetylation, and PARP1 mono-ADP ribosylation). Progerin may interfere with SIRT6 activation and SIRT6 mediated molecular events in response to DNA damage.

Griseofulvin derivatives have been developed that have anti-inflammatory activity and have been disclosed for use in treating inflammatory diseases, including central inflammatory disease such as neurodegenerative diseases. See WO 2017/170623 and WO 2019/065928.

There is a need in the art for effective treatments for natural aging and premature aging disorders.

### SUMMARY OF THE INVENTION

The present invention is based on the relationship between SIRT6, lamin A, progerin, and aging and the discovery that the compounds disclosed in WO 2017/170623 and WO 2019/065928 are potent activators of SIRT6. In particular, compounds that can activate SIRT6 in a subject that has a mutation in lamin A and/or increased levels of progerin (e.g., as a result of natural aging or a disorder) may permit enhanced function of SIRT6, which may lead to enhanced DNA repair and/or telomere maintenance. Thus, a SIRT6 activator may be effective to treat, slow down, or prevent age-related diseases and premature aging disorders.

Thus, one aspect of the invention relates to a method of treating, delaying the onset of, slowing the progression of, or reducing the severity of a premature aging disorder in a subject in need thereof, comprising administering to the subject a therapeutically effective amount of the compound of Formula 1 or a pharmaceutically acceptable salt thereof:

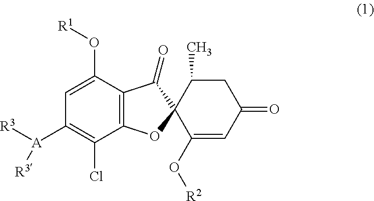

wherein:
R′ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH═, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
Substituent group Y is a C1-C6 alkyl group, a C1-C6 alkoxy group, a halogen atom, or a hydroxy group,
thereby treating, delaying the onset of, slowing the progression of, or reducing the severity of the premature aging disorder.

In some embodiments, the premature aging disorder is Hutchinson—Gilford progeria syndrome, Werner syndrome, Bloom syndrome, Rothmund—Thomson syndrome, Cockayne syndrome, xeroderma pigmentosum, trichothiodystrophy, combined xeroderma pigmentosum-Cockayne syndrome, or restrictive dermopathy.

Another aspect of the invention relates to a method of treating, delaying the onset of, slowing the progression of, or reducing the severity of an age-related disease in a subject in need thereof, comprising administering to the subject a therapeutically effective amount of a compound of Formula 1 or a pharmaceutically acceptable salt thereof:

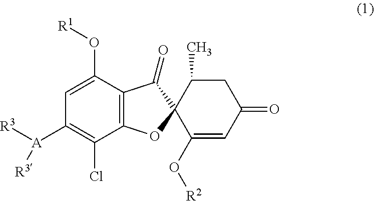

wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH═, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
Substituent group Y is a C1-C6 alkyl group, a C1-C6 alkoxy group, a halogen atom, or a hydroxy group,
thereby treating, delaying the onset of, slowing the progression of, or reducing the severity of the age-related disease.

In some embodiments, the age-related disease is type 2 diabetes, neurodegenerative disease, cancer, cardiovascular disease, obesity, increased cholesterol levels, hypertension, ocular disorders, cataracts, glaucoma, osteoporosis, blood clotting disorders, arthritis, hearing loss, stroke, Alzheimer's disease, osteoarthritis, rheumatoid arthritis, chronic obstructive pulmonary disease, fatty liver disease, or nonalcoholic steatohepatitis.

The invention additionally relates to use of the compounds of the invention to treat, delay the onset of, slow the progression of, or reduce the severity of a premature aging disorder.

The invention further relates to use of the compounds of the invention to treat, delay the onset of, slow the progression of, or reduce the severity of an age-related disease.

These and other aspects of the invention are set forth in more detail in the description of the invention below.

### DETAILED DESCRIPTION OF EMBODIMENTS OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, nucleotide sequences, amino acid sequences and other references mentioned herein are incorporated by reference in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

Furthermore, the term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

As used herein, the transitional phrase “consisting essentially of” is to be interpreted as encompassing the recited materials or steps and those that do not materially affect the basic and novel characteristic(s) of the claimed invention. Thus, the term “consisting essentially of” as used herein should not be interpreted as equivalent to “comprising.”

By the term “treat,” “treating,” or “treatment of” (or grammatically equivalent terms) is meant to reduce or to at least partially improve or ameliorate the severity of the subject's condition and/or to alleviate, mitigate or decrease in at least one clinical symptom and/or to delay the progression of the condition.

As used herein, the term “prevent,” “prevents,” or “prevention” (and grammatical equivalents thereof) means to delay or inhibit the onset of a disease. The terms are not meant to require complete abolition of disease, and encompass any type of prophylactic treatment to reduce the incidence of the condition or delays the onset of the condition.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21^{st} ed. 2005). Exemplary pharmaceutically acceptable carriers for the compositions of this invention include, but are not limited to, sterile pyrogen-free water and sterile pyrogen-free physiological saline solution.

“Concurrently” means sufficiently close in time to produce a combined effect (that is, concurrently can be simultaneously, or it can be two or more events occurring within a short time period before or after each other). In some embodiments, the administration of two or more compounds “concurrently” means that the two compounds are administered closely enough in time that the presence of one alters the biological effects of the other. The two compounds can be administered in the same or different formulations or sequentially. Concurrent administration can be carried out by mixing the compounds prior to administration, or by administering the compounds in two different formulations, for example, at the same point in time but at different anatomic sites or using different routes of administration.

One aspect of the invention relates to a method of treating, delaying the onset of, slowing the progression of, or reducing the severity of a premature aging disorder in a subject in need thereof, comprising administering to the subject a therapeutically effective amount of the compound of Formula 1 or a pharmaceutically acceptable salt thereof:

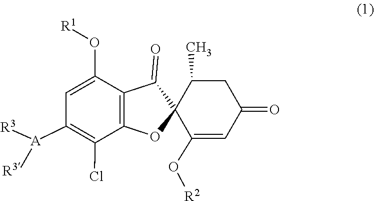

wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH═, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
Substituent group Y is a C1-C6 alkyl group, a C1-C6 alkoxy group, a halogen atom, or a hydroxy group,
thereby treating, delaying the onset of, slowing the progression of, or reducing the severity of the premature aging disorder.

In some embodiments, the premature aging disorder incudes, but is not limited to Hutchinson—Gilford progeria syndrome, Werner syndrome, Bloom syndrome, Rothmund—Thomson syndrome, Cockayne syndrome, xeroderma pigmentosum, trichothiodystrophy, combined xeroderma pigmentosum-Cockayne syndrome, or restrictive dermopathy. In certain embodiments, the premature aging disorder is Hutchinson—Gilford progeria syndrome.

In certain embodiments, the methods of the invention may treat, delay the onset of, slow the progression of, or reduce the severity of one or more symptoms of the premature aging disorder, e.g., HGPS. Symptoms may include, without limitation, failure to thrive, maldevelopment, cardiovascular disease (e.g., atherosclerosis, atherosclerotic plaques in large or small arteries, interstitial fibrosis, stenosis, or paucity of medial smooth muscle cells), abnormal bone density, distal bone resorption, osteoporosis, or decreased adipose tissue.

In certain embodiments, the methods of the invention may treat, delay the onset of, slow the progression of, or reduce the severity of any disease or disorder associated with a mutation in lamin A, referred to as laminopathies. There currently are eight diseases in addition to HGPS that are laminopathies, including such disorders as Emery-Dreifuss muscular dystrophy, mandibuloacral dysplasia, atypical Werner's syndrome, dilated cardiomyopathy-type 1A, restrictive dermopathy, and Dunnigan-type familial partial lipodystrophy.

A further aspect of the invention relates to a method of treating, delaying the onset of, slowing the progression of, or reducing the severity of an age-related disease in a subject in need thereof, comprising administering to the subject a therapeutically effective amount of a compound of Formula 1 or a pharmaceutically acceptable salt thereof:

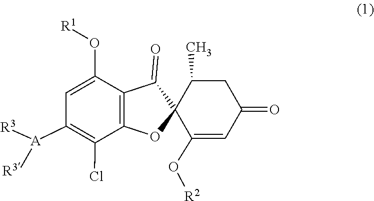

wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH═, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
Substituent group Y is a C1-C6 alkyl group, a C1-C6 alkoxy group, a halogen atom, or a hydroxy group,
thereby treating, delaying the onset of, slowing the progression of, or reducing the severity of the age-related disease.

In some embodiments, the age-related disease is type 2 diabetes, neurodegenerative disease (e.g., Alzheimer's disease, Parkinson's disease, Huntington's diseases, amyotrophic lateral sclerosis, and multiple system atrophy), cancer, cardiovascular disease (e.g., coronary heart diseases, cardiomyopathy, hypertensive heart diseases, cardiac dysrhythmias, endocarditis, cardiomegaly, myocarditis, calcular heart diseases, cerebrovascular disease, and peripheral arterial disease), obesity, increased cholesterol levels, hypertension, ocular disorders, cataracts, glaucoma, osteoporosis, blood clotting disorders, arthritis, hearing loss, stroke, Alzheimer's disease, Parkinson's disease, amyotrophic lateral sclerosis, Huntington's chorea, and Lewy Body disease, osteoporosis, osteoarthritis, rheumatoid arthritis, intervertebral disc degeneration, chronic obstructive pulmonary disease, fatty liver disease, or nonalcoholic steatohepatitis.

Indications of age-related diseases that may be treatable by the methods of the invention include, without limitation, epidermal atrophy, epidermal hyperpigmentation, rhytid (wrinkles), photoaging of the skin, alopecia, hearing loss, visual impairment, cerebral atrophy, cognitive deficits, trembling, ataxia, areflexia, cerebellar degeneration, hypertension, renal insufficiency, renal acidosis, incontinence, endocrinopathies, diabetes, decreased liver function, hypoalbuminemia, hepatic accumulation of glycogen and triglycerides, anemia, bone marrow degeneration, osteopenia, osteoporosis, kyphosis, degenerative joint disease, intervertebral disc degeneration, peripheral neuropathy, impaired wound healing, increased cellular senescence, retinal degeneration, motor neuron degeneration, cerebral lacunae, white matter degeneration, sarcopenia, muscle weakness, dystonia, increased peroxisome biogenesis, increased apoptosis, decreased cellular proliferation, cachexia, and decreased lifespan.

In some embodiments, the compound of Formula 1 is any compound selected from the following group:
(2S,5′R)-7-chloro-6-(5-ethyl-1,3,4-oxadiazol-2-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro [benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl] spiro [benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro [benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro [benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(1H-pyrazol-5-yl) spiro [benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro [benzofuran-2,4′-cyclohex-2-ene]-1′,3-dione;
(2S,5′R)-7-chloro-6-(1,8-dioxa-2-azaspiro[4.5] dec-2-en-3-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5] dec-2-en-3-yl) spiro [benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro [benzofuran-2,4′-cyclohex-2-ene]-1′,3-dione;
(2S,5′R)-7-chloro-3′,4-dimethoxy-6-(6-methoxy-3-pyridyl)-5′-methyl-spiro [benzofuran-2,4′-cyclohex-2-ene]-1′,3-dione; or
(2S,5′R)-7-chloro-3′,4-dimethoxy-5′-methyl-6-(3-pyridyl) spiro [benzofuran-2,4′-cyclohex-2-ene]-1′,3-dione.

In some embodiments, the compound of Formula 1 is a compound of Formula 1′ or a pharmacologically acceptable salt thereof:

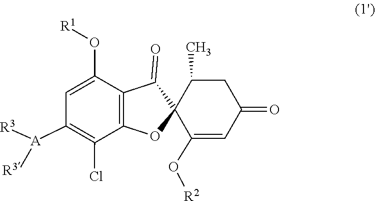

wherein:
R¹ is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X.
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, or a single bond, wherein when it is a single bond, one or the other of R³ and R^{3′} is not present,
R³ and R^{3′} are each independently a hydrogen atom, a halogen atom, a cyano group, a hydroxy group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from the substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group, and
Substituent group Y is a C1-C6 alkyl group, a C1-C6 alkoxy group, a halogen atom, or a hydroxy group.

In some embodiments of the compound of Formula 1 or Formula 1′, R¹ is a C1-C6 alkyl group, R² is a C1-C6 alkyl group, A is a 5-membered aromatic heterocyclic ring, and R³ and R^{3′} are each independently a hydrogen or a C1-C6 alkyl group.

In some embodiments of the compound of Formula 1 or Formula 1′, R¹ is a methyl group, an ethyl group, or a hydroxyethyl group.

In some embodiments of the compound of Formula 1 or Formula 1′, R² is a methyl group.

In some embodiments of the compound of Formula 1 or Formula 1′, A is a 5-membered aromatic heterocyclic ring, R³ is a methyl group, an ethyl group, a hydroxy C1-C3 alkyl group,

or a methoxy C1-C3 alkyl group, and R^{3′} is a hydrogen atom.

In some embodiments, the compound of Formula 1 is a compound of a Formula 1″ or a pharmacologically acceptable salt thereof:

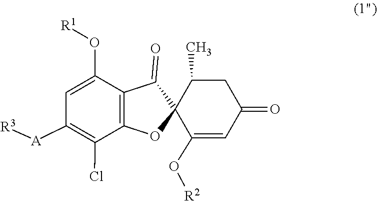

wherein R¹ is a methyl group or an ethyl group;
R² is a methyl group;
A is any ring selected from the following group:

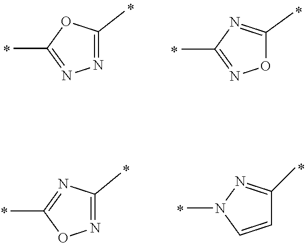

* indicates a binding group; and
R³ is a methyl group or an ethyl group.

In some embodiments, the compound of Formula 1′ is any compound selected from the following group:
(2S,5′R)-7-chloro-6-(2-hydroxyethoxy)-3′,4-dimethoxy-5′-methyl-spiro [benzofuran-2,4′-cyclohex-2-ene]-1′,3-dione;
(2S,5′R)-7-chloro-3′,4-dimethoxy-6-(2-methoxyethoxy)-5′-methyl-spiro [benzofuran-2,4′-cyclohex-2-ene]-1′,3-dione;
(2S,5′R)-7-chloro-3′,4-dimethoxy-5′-methyl-6-(1-methylpyrazol-3-yl) spiro [benzofuran-2,4′-cyclohex-2-ene]-1′,3-dione;
(2S,5′R)-7-chloro-6-(1-ethylpyrazol-3-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(5-methyl-1,3,4-oxadiazol-2-yl) spiro [benzofuran-2,4′-cyclohex-2-ene]-1′,3-dione;
(2S,5′R)-7-chloro-3′,4-dimethoxy-5′-methyl-6-(3-methyl-1,2,4-oxadiazol-5-yl) spiro [benzofuran-2,4′-cyclohex-2-ene]-1′,3-dione;
(2S,5′R)-7-chloro-3′,4-dimethoxy-5′-methyl-6-(5-methyl)-1,2,4-oxadiazol-3-yl) spiro [benzofuran-2,4′-cyclohex-2-ene]-1′,3-dione;
(2S,5′R)-7-chloro-6-[5-(1-hydroxy-1-methyl-ethyl)-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-6-[5-[(1S)-1-hydroxyethyl]-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-6-[5-[(1R)-1-hydroxyethyl]-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-4-ethoxy-6-[5-(1-hydroxy-1-methyl-ethyl)-1,3,4-oxadiazol-2-yl]-3′-methoxy-5′-methyl-spiro [benzofuran-2,4′-cyclohex-2-ene]-1′,3-dione;
(2S,5′R)-7-chloro-4-ethoxy-6-[5-[(1S)-1-hydroxyethyl]-1,3,4-oxadiazol-2-yl]-3′-methoxy-5′-methyl-spiro [benzofuran-2,4′-cyclohex-2-ene]-1′,3-dione;
(2S,5′R)-7-chloro-6-[3-(1-hydroxyethyl)-1,2,4-oxadiazol-5-yl]-3′,4-dimethoxy-5′-meth yl-spiro [benzofuran-2,4′-cyclohex-2-ene]-1′,3-dione;
(2S,5′R)-7-chloro-4-(2-hydroxyethoxy)-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-4-(2-hydroxyethoxy)-3′-methoxy-5′-methyl-6-(3-methyl-1,2,4-oxadiazol-5-yl) spiro [benzofuran-2,4′-cyclohex-2-ene]-1′,3-dione;
(2S,5′R)-7-chloro-4-(2-hydroxyethoxy)-3′-methoxy-5′-methyl-6-(5-methyl-1,2,4-oxadiazol-3-yl) spiro [benzofuran-2,4′-cyclohex-2-ene]-1′,3-dione;
(2S,5′R)-7-chloro-6-(5-ethyl-1,3,4-oxadiazol-2-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro [benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl] spiro [benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro [benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro [benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(1H-pyrazol-5-yl) spiro [benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro [benzofuran-2,4′-cyclohex-2-ene]-1′,3-dione;
(2S,5′R)-7-chloro-6-(1,8-dioxa-2-azaspiro[4.5] dec-2-en-3-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5] dec-2-en-3-yl) spiro [benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro [benzofuran-2,4′-cyclohex-2-ene]-1′,3-dione;
(2S,5′R)-7-chloro-3′,4-dimethoxy-6-(6-methoxy-3-pyridyl)-5′-methyl-spiro [benzofuran-2,4′-cyclohex-2-ene]-1′,3-dione;
(2S,5′R)-7-chloro-3′,4-dimethoxy-5′-methyl-6-(3-pyridyl) spiro [benzofuran-2,4′-cyclohex-2-ene]-1′,3-dione; or
(2S,5′R)-7-chloro-3′,4,6-trimethoxy-5′-methyl-spiro [benzofuran-2,4′-cyclohex-2-ene]-1′,3-dione.

In one embodiment, the compound is (2S,5′R)-7-chloro-6-(1-ethylpyrazol-3-yl)-3′,4-dimethoxy-5′-methyl-spiro [benzofuran-2,4′-cyclohex-2-ene]-1′,3-dione or a pharmacologically acceptable salt thereof.

In one embodiment, the compound is (2S,5′R)-7-chloro-3′,4-dimethoxy-5′-methyl-6-(5-methyl-1,3,4-oxadiazol-2-yl) spiro [benzofuran-2,4′-cyclohex-2-ene]-1′,3-dione or a pharmacologically acceptable salt thereof.

In one embodiment, the compound is (2S,5′R)-7-chloro-6-(5-ethyl-1,3,4-oxadiazol-2-yl)-3′,4-dimethoxy-5′-methyl-spiro [benzofuran-2,4′-cyclohex-2-ene]-1′,3-dione or a pharmacologically acceptable salt thereof.

In one embodiment, the compound is (2S,5′R)-7-chloro-6-[3-(1-hydroxy-1-methyl-ethyl)-1,2,4-oxadiazol-5-yl]-3′,4-dimethoxy-5′-methyl-spiro [benzofuran-2,4′-cyclohex-2-ene]-1′,3-dione or a pharmacologically acceptable salt thereof.

In one embodiment, the compound is (2S,5′R)-7-chloro-4-ethoxy-3′-methoxy-5′-methyl-6-(5-methyl-1,3,4-oxadiazol-2-yl) spiro [benzofuran-2,4′-cyclohex-2-ene]-1′,3-dione or a pharmacologically acceptable salt thereof.

In certain embodiments, the 5-membered aromatic heterocyclic ring for A is the same as described above, but more preferably, it represents the following 5-membered ring. (It should be noted that in this case, R^{3′} is not present.)

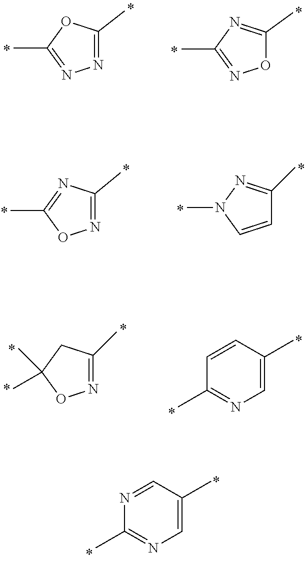

wherein * indicates a binding group.

In the present specification, the “5-membered aromatic heterocyclic ring” is a monocyclic 5-membered aromatic heterocyclic ring containing one to four atoms selected from the group consisting of a nitrogen atom, an oxygen atom, and a sulfur atom. For example, rings such as those shown below are included.

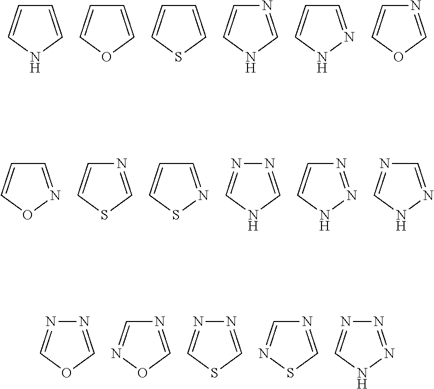

In the present specification, the “6-membered aromatic heterocyclic ring” is a monocyclic 6-membered aromatic heterocyclic ring containing one to four atoms selected from the group consisting of a nitrogen atom, an oxygen atom, and a sulfur atom. For example, rings such as those shown below are included.

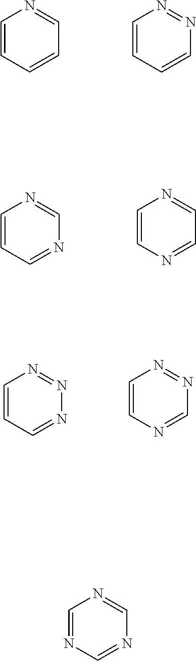

In the present specification, the “8-10 membered condensed aromatic heterocyclic ring” is an 8-10 membered condensed aromatic heterocyclic ring containing one to four atoms selected from the group consisting of a nitrogen atom, an oxygen atom, and a sulfur atom. For example, rings such as those shown below are included.

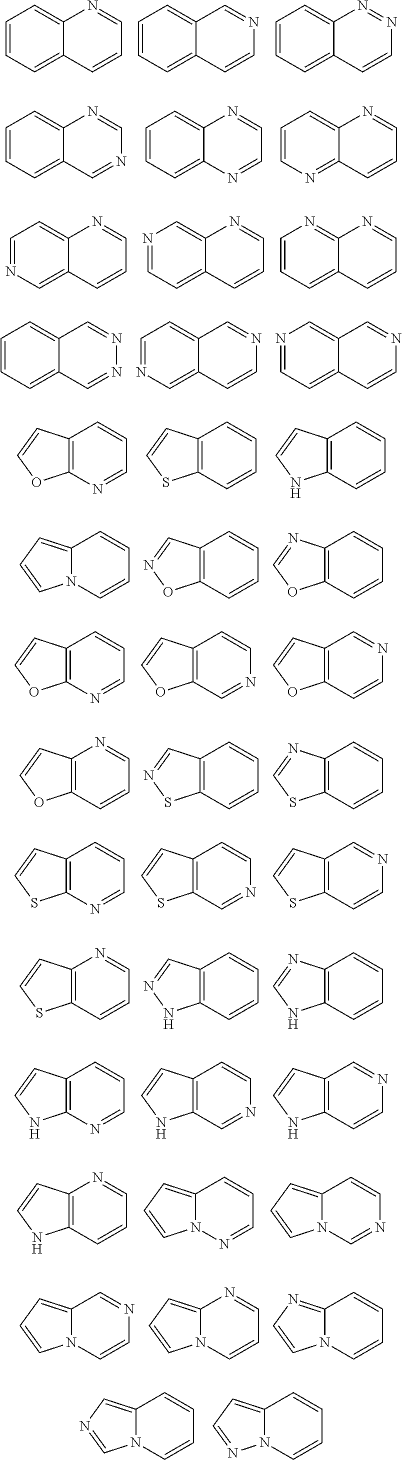

In the present specification, the “5-7 membered unsaturated heterocyclic ring” is a ring in which a monocyclic 5-7 membered saturated heterocyclic ring is partially oxidized or a ring in which an aromatic heterocyclic ring is partially reduced containing one to four atoms selected from the group consisting of a nitrogen atom, an oxygen atom, and a sulfur atom. For example, rings such as those shown below are included.

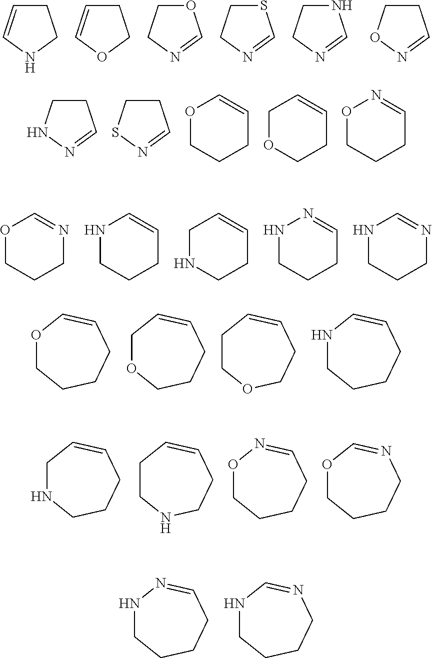

In the present specification, the “4-7 membered saturated heterocyclic ring” is a monocyclic 4-7 membered saturated heterocyclic ring containing one to four atoms selected from the group consisting of a nitrogen atom, an oxygen atom, and a sulfur atom. For example, rings such as those shown below are included.

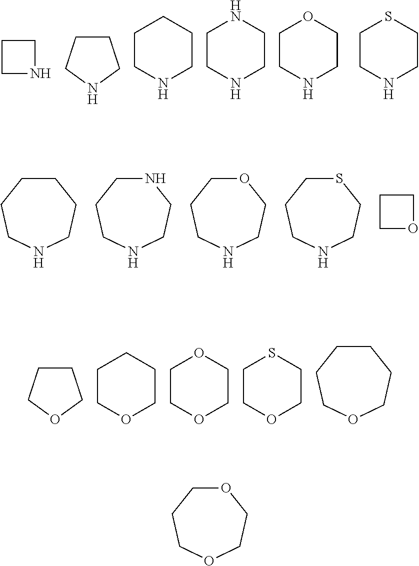

The “halogen atom” in the present specification is a fluorine atom, a chlorine atom, a bromine atom, or an iodine atom, and is preferably a fluorine atom or a chlorine atom.

The “C1-C6 alkyl group” in the present specification is a linear or branched alkyl group having one to six carbon atoms. Examples thereof include a methyl group, an ethyl group, a 1-propyl group, an isopropyl group, a 1-butyl group, a 2-butyl group, a 2-methyl-1-propyl group, a 2-methyl-2-propyl group, a 1-pentyl group, a 2-pentyl group, a 3-pentyl group, a 2-methyl-2-butyl group, a 3-methyl-2-butyl group, a 1-hexyl group, a 2-hexyl group, a 3-hexyl group, a 2-methyl-1-pentyl group, a 3-methyl-1-pentyl group, a 2-ethyl-1-butyl group, a 2,2-dimethyl-1-butyl group, and a 2,3-dimethyl-1-butyl group, and it is preferably a methyl group or an ethyl group.

The “C2-C6 alkenyl group” in the present specification is a linear or branched alkenyl group having two to six carbon atoms, and it may have one or two or more carbon-carbon double bonds. For example, it is a vinyl group, a 2-propenyl (allyl) group, a 2-butenyl group, a 2-pentenyl group, a 3-methyl-2-butenyl group, a 2-hexenyl group, or a 3-methyl-2-pentenyl group, and preferably, it is a vinyl group or an allyl group.

The “C2-C6 alkynyl group” in the present specification is a linear or branched alkynyl group having two to six carbon atoms, and it may have one or two or more carbon-carbon triple bonds. For example, it is an ethynyl group, a 1-propynyl group, a 2-propynyl group, a 1-butynyl group, a 2-butynyl group, a 1-pentynyl group, a 2-pentynyl group, or 1-hexynyl group, and it is preferably an ethynyl group or a 1-propynyl group.

The “C1-C6 alkoxy group” in the present specification is a group in which an oxygen atom is bonded to a C1-C6 alkyl group. Examples thereof include a methoxy group, an ethoxy group, a 1-propoxy group, a 2-propoxy group, a 1-butoxy group, a 2-butoxy group, a 2-methyl-1-propoxy group, a 2-methyl-2-propoxy group, a 1-pentyloxy group, a 2-pentyloxy group, a 3-pentyloxy group, a 2-methyl-2-butoxy group, a 3-methyl-2-butoxy group, a 1-hexyloxy group, a 2-hexyloxy group, a 3-hexyloxy group, a 2-methyl-1-pentyloxy group, and a 3-methyl-1-pentyloxy group. Preferably, it is a methoxy group, an ethoxy group, a 1-propoxy group, or a 2-propoxy group.

The “C3-C6 cycloalkyl group” in the present specification is a cyclic alkyl group having three to six carbon atoms, and it is preferably a cyclopropyl group, a cyclobutyl group, a cyclopentyl group, or a cyclohexyl group.

The “hydroxy C1-C6 alkyl group” in the present specification is a group in which a hydroxyl group is bonded to a C1-C6 alkyl group. For example, it is a hydroxymethyl group or a hydroxyethyl group.

The “C1-C6 alkoxy C1-C6 alkyl group” in the present specification is a group in which a C1-C6 alkoxy is bonded to a C1-C6 alkyl group. Examples thereof include a methoxymethyl group, a methoxyethyl group, an ethoxymethyl group, and an ethoxyethyl group.

The “C1-C6 haloalkyl group” in the present specification is a group in which a halogen atom is bonded to a C1-C6 alkyl group. Examples thereof include a fluoromethyl group, a difluoromethyl group, a dichloromethyl group, a dibromomethyl group, a trifluoromethyl group, a trichloromethyl group, a 2-fluoroethyl group, a 2-bromoethyl group, a 2-chloroethyl group, a 2-iodoethyl group, a 2,2-difluoroethyl group, a 2,2,2-trifluoroethyl group, a trichloroethyl group, a pentafluoroethyl group, a 3-fluoropropyl group, a 3-chloropropyl group, and a 4-fluorobutyl group. It is preferably a trifluoromethyl group.

The “C3-C6 halocycloalkyl group” in the present specification is a group in which a halogen atom is bonded to a C3-C6 cycloalkyl group, and examples thereof include a fluorocyclopropyl group, a fluorocyclobutyl group, a fluorocyclopentyl group, and a fluorocyclohexyl group.

The “C1-C6 haloalkoxy group” in the present specification is a group in which a halogen atom is bonded to a C1-C6 alkoxy group, and examples thereof include a fluoromethoxy group, a difluoromethoxy group, a dichloromethoxy group, a dibromomethoxy group, a trifluoromethoxy group, a trichloromethoxy group, a 2-fluoroethoxy group, a 2-bromoethoxy group, a 2-chloroethoxy group, a 2-iodoethoxy group, a 2,2-difluoroethoxy group, a 2,2,2-trifluoroethoxy group, a trichloroethoxy group, a pentafluoroethoxy group, a 3-fluoropropoxy group, a 3-chloropropoxy group, and a 4-fluorobutoxy group. It is preferably a trifluoromethoxy group.

The “C3-C6 cycloalkoxy group” in the present specification is a group in which a C3-C6 cycloalkyl group is bonded to an oxygen atom, and it is preferably a cyclopropyloxy group, a cyclobutyloxy group, a cyclopentyloxy group, or a cyclohexyloxy group.

The “C3-C6 halocycloalkoxy group” in the present specification is a group in which a C3-C6 halocycloalkyl group is bonded to an oxygen atom, and examples thereof include a fluorocyclopropoxy group, a fluorocyclobutoxy group, a fluorocyclopentyloxy group, and a fluorocyclohexyloxy group.

The “5-membered aromatic heterocyclic oxy group” in the present specification is a group in which a 5-membered aromatic heterocyclic ring is bonded to an oxygen atom.

The “6-membered aromatic heterocyclic oxy group” in the present specification is a group in which a 6-membered aromatic heterocyclic ring is bonded to an oxygen atom.

The “4-7 membered saturated heterocyclic oxy group” in the present specification is a group in which a 4-7 membered saturated heterocyclic ring is bonded to an oxygen atom.

The “C1-C6 alkoxycarbonyl group” in the present specification is a group in which a C1-C6 alkoxy group is bonded to a carbonyl group, and examples thereof include a methoxycarbonyl group, an ethoxycarbonyl group, and a propoxycarbonyl group.

The “C3-C6 cycloalkoxycarbonyl group” in the present specification is a group in which a C3-C6 cycloalkoxy group is bonded to a carbonyl group, and it is preferably a cyclopropyloxycarbonyl group, a cyclobutyloxycarbonyl group, a cyclopentyloxycarbonyl group, or a cyclohexyloxycarbonyl group.

The “C1-C6 alkyl carbonyl group” in the present specification is a group in which a C1-C6 alkyl group is bonded to a carbonyl group, and examples thereof include a methyl carbonyl group, an ethyl carbonyl group, or a propyl carbonyl group.

The “mono (C1-C6 alkyl) aminocarbonyl group” in the present specification is a group in which one C1-C6 alkyl group is bonded to the amino group of an aminocarbonyl group, and it is preferably a methylaminocarbonyl group, an ethylaminocarbonyl group, or a propylaminocarbonyl group.

The “di (C1-C6 alkyl) aminocarbonyl group” in the present specification is a group in which two C1-C6 alkyl groups are bonded to the amino group of an aminocarbonyl group, and it is preferably a dimethylaminocarbonyl group, a diethylaminocarbonyl group, or a dipropylaminocarbonyl group.

The “mono (C1-C6 alkyl) aminosulfonyl group” in the present specification is a group in which one C1-C6 alkyl group is bonded to the amino group of an aminosulfonyl group, and it is preferably a methylaminosulfonyl group, an ethylaminosulfonyl group, or a propylaminosulfonyl group

The “di (C1-C6 alkyl) aminosulfonyl group” in the present specification is a group in which two C1-C6 alkyl groups are bonded to the amino group of the aminosulfonyl group, and it is preferably a dimethylaminosulfonyl group, a diethylaminosulfonyl group, or a dipropylaminosulfonyl group.

The “mono (C1-C6 alkyl) amino group” in the present specification is a group in which one C1-C6 alkyl group is bonded to an amino group, and it is preferably a methylamino group, an ethylamino group, or a propylamino group.

The “di (C1-C6 alkyl) amino group” in the present specification is a group in which two C1-C6 alkyl groups are bonded to an amino group, and it is preferably a dimethylamino group, a diethylamino group, or a dipropyl amino group.

The “C1-C6 alkoxycarbonylamino group” in the present specification is a group in which a C1-C6 alkoxycarbonyl group is bonded to an amino group, and for example, it is a methoxycarbonylamino group, an ethoxycarbonylamino group, or a propoxycarbonylamino group.

The “mono (C1-C6 alkyl) aminocarbonylamino group” in the present specification is a group in which a mono (C1-C6 alkyl) aminocarbonyl group is bonded to an amino group, and it is preferably a methylaminocarbonylamino group, an ethylaminocarbonylamino group, or a propylaminocarbonylamino group.

The “di (C1-C6 alkyl) aminocarbonylamino group” in the present specification is a group in which a di (C1-C6 alkyl) aminocarbonyl group is bonded to an amino group, and it is preferably a dimethylaminocarbonylamino group, a diethylaminocarbonylamino group, or a dipropylaminocarbonylamino group.

The “5-membered aromatic heterocyclic carbonylamino group” in the present specification is a group in which a 5-membered aromatic heterocyclic carbonyl group is bonded to an amino group.

The “6-membered aromatic heterocyclic carbonylamino group” in the present specification is a group in which a 6-membered aromatic heterocyclic carbonyl group is bonded to an amino group.

The “C1-C6 alkylsulfonylamino group” in the present specification is a group in which a C1-C6 alkyl group is bonded to the sulfonyl group of a sulfonylamino group, and it is preferably a methylsulfonylamino group, an ethylsulfonylamino group, or a propylsulfonylamino group.

The “pharmaceutically acceptable salt” indicates a salt that can be used as a pharmaceutical. When the compound has an acidic group or a basic group it can be converted to a basic salt or an acidic salt by reacting with a base or an acid to form a salt thereof.

The pharmaceutically acceptable “basic salt” of the compound preferably includes an alkali metal salt such as a sodium salt, a potassium salt, and a lithium salt; an alkaline earth metal salt such as a magnesium salt and a calcium salt; organic base salts such as an N-methyl morpholine salt, a triethylamine salt, a tributylamine salt, a diisopropylethylamine salt, a dicyclohexylamine salt, an N-methylpiperidine salt, a pyridine salt, a 4-pyrrolidinopyridine salt, and a picoline salt; and an amino acid salt such as glycine salt, a lysine salt, an arginine salt, an ornithine salt, a glutamate, and an aspartate, and it is preferably an alkali metal salt.

The pharmaceutically acceptable “acidic salt” of the compound preferably includes an inorganic acid salt such as a hydrohalide such as a hydrofluoride, a hydrochloride, a hydrobromide, and a hydroiodide, a nitrate, a perchlorate, a sulfate, and a phosphate; an organic salt such as a lower alkanesulfonate such as methanesulfonate, trifluoromethanesulfonate, and ethanesulfonate, an aryl sulfonate such as a benzenesulfonates, and a p-toluene sulfonate, an acetate, a malate, a fumarate, a succinate, a citrate, an ascorbate, a tartrate, an oxalate, a maleate, and the like; and an amino acid salt such as glycine salt, a lysine salt, an arginine salt, an ornithine salt, a glutamate, and an aspartate, and it is most preferably a hydrohalide (in particular, a hydrochloride).

The compound of the present invention or the pharmaceutically acceptable salt thereof may absorb moisture, adhere to adsorbed water, or become a hydrate by leaving in the air or recrystallization. The present invention also encompasses compounds of such various hydrates, solvates, and crystalline polymorphs.

The compounds of the present invention, their pharmaceutically acceptable salts or solvates thereof, depending on the type and combination of substituents, may have various isomers such as geometric isomers such as a cis isomer and a trans isomer, tautomers, or optical isomers such as a d isomer and an l isomer, while the compounds include those all isomers, stereoisomers, and mixtures of these isomers and stereoisomers in any ratio unless otherwise specified. Mixtures of these isomers may be resolved by known resolution means.

[The compounds of the present invention also include labels, that is, a compound in which one or more atoms of the compounds are substituted with an isotope (for example, 2H, 3H, 13C, 14C, 35S, and the like).

In addition, the present invention also encompasses a prodrug. The prodrug is a compound having a group which can be converted to an amino group, a hydroxyl group, a carboxyl group, or the like of the compound by hydrolysis or under physiological conditions, and as a group forming such a prodrug, it is a group described in Prog. Med., Vol. 5, pp. 2157 to 2161 (1985) or the like. As the prodrug, more specifically, when an amino group is present in the compound, a compound in which the amino group is acylated, alkylated, or phosphorylated (for example, it is a compound in which the amino group is eicosanoylated, alanylated, pentylaminocarbonylated, (5-methyl-2-oxo-1,3-dioxolen-4-yl) methoxycarbonylated, tetrahydrofuranylated, pyrrolidinyl methylated, pivaloyloxymethylatied, or tert-butylated, or the like) and the like are included, and when a hydroxyl group is present in the compound, a compound in which the hydroxyl group is acylated, alkylated, phosphorylated, or borated (for example, it is a compound in which the hydroxyl group is acetylated, palmitoylated, propanoylated, pivaloylated, succinylated, fumarylated, alanylated, or dimethylaminomethyl carbonylated, or the like.) and the like are included. In addition, when a carboxy group is present in the compound, a compound in which the carboxy group is esterified or amidated (for example, it is a compound in which the carboxy group is ethyl esterified, phenyl esterified, carboxymethyl esterified, dimethylaminomethyl esterified, pivaloyloxymethyl esterified, ethoxycarbonyloxyethyl esterified, amidated, or methylamidated, or the like.), and the like are included.

[The compounds of the present invention may be produced by synthetic methods known in the art and as described in WO 2017/170623 and WO 2019/065928, incorporated by reference herein in their entirety.

Administration of the compounds of the present invention may be carried out by any form of oral administration by a tablet, a pill, a capsule, a granule, a powder, a solution, or the like, or by any form of parenteral administration by an injection for intra-articular, intravenous, intramuscular, or the like, a suppository, an eye drop, an eye ointment, a transdermal solution, an ointment, a transdermal patch, a transmucosal solution, a transmucosal patch, an inhalant, or the like.

As a solid composition for oral administration, a tablet, a powder, a granule, and the like are used. Such a solid composition is composed of one or more active ingredients and at least one inert excipient such as lactose, mannitol, glucose, hydroxypropyl cellulose, microcrystalline cellulose, starch, polyvinyl pyrrolidone, magnesium metasilicate aluminate, and/or the like. The solid composition may contain, according to a conventional method, one or more of an inert additive such as a lubricant such as magnesium stearate, a disintegrant such as sodium carboxymethyl starch, a stabilizer, and a solubilizer. The tablet or pill may be coated with a sugar coating or a film of a substance soluble in the stomach or intestine, if necessary.

As a liquid composition for oral administration, a pharmaceutically acceptable emulsion, solution, suspension, syrup, elixir, or the like is used. To such a liquid composition, it is possible to add a generally used inert diluent such as purified water or ethanol. The liquid composition may contain, in addition to an inert diluent, one or more of a solubilizer, an adjuvant such as a wetting agent, a sweetening agent, a flavoring agent, a fragrance, and a preservative.

As an injection for parenteral administration, a sterile aqueous or non-aqueous solution, a suspension or an emulsion, and the like are used. The aqueous solvent includes, for example, distilled water for injection, physiological saline, and the like. The non-aqueous solvents include, for example, propylene glycol, polyethylene glycol, and vegetable oil such as olive oil, alcohols such as ethanol, Polysorbate 80, and the like. Such an injection composition may further contain a one or more of a tonicity agent, a preservative, a wetting agent, an emulsion, a dispersing agent, a stabilizer, or a solubilizer. These injection compositions can be sterilized by, for example, filtration through a bacteria retention filter, application of a bactericide, or irradiation. In addition, these injection compositions may be used by producing a sterile solid composition and dissolved or suspended in sterile water or a sterile solvent for injection prior to use.

As an external preparation, an ointment, a plaster, a cream, a jelly, a cataplasm, a spray, a lotion, an eye drop, an eye ointment, and the like are used. These external preparations include generally used ointment bases, lotion bases, aqueous or non-aqueous solutions, suspensions, emulsions, and the like. For example, as an ointment or lotion base, polyethylene glycol, propylene glycol, white petrolatum, bleached beeswax, polyoxyethylene hydrogenated castor oil, glycerin monostearate, stearyl alcohol, cetyl alcohol, lauromacrogol, sorbitan sesquioleate, and the like are used.

A transmucosal agent such as an inhalant and a transnasal agent are used in solid, liquid, or semisolid form, and it may be produced according to a conventionally known method. For example, a known excipient, and furthermore, one or more of a pH adjuster, a preservative, a surfactant, a lubricant, a stabilizer, a thickener, and the like may be added as appropriate. With these transmucosal agents, devices appropriate for inhalation or insufflation may be used as the method of administration. For example, the compound may be administered alone or as a powder of a formulated mixture, or as a solution or suspension in combination with a pharmaceutically acceptable carrier, using known devices and nebulizers, such as metered dose inhalation devices. A dry powder inhaler or the like may be for single or multiple administration, and a dry powder or powder containing capsule may be also used. Alternatively, an appropriate ejector may be used. For example, it may be in the form of a pressurized aerosol spray or the like using a suitable gas such as chlorofluoroalkane, hydrofluoroalkane, or carbon dioxide.

In the case of normal oral administration, the appropriate daily dose is about 0.001 to 100 mg/kg, preferably 0.1 to 30 mg/kg, and more preferably 0.1 to 10 mg/kg of body weight. This is administered in one dose or separated into two or more doses. When administered intravenously, the appropriate daily dose is about 0.0001 to 10 mg/kg of body weight, which is administered once or separated into several times a day. In addition, as a transmucosal agent, about 0.001 to 100 mg/kg of body weight is administered once or separated into several times a day. The dose is appropriately determined depending on the individual case in consideration of symptoms, age, sex, and the like.

In the methods of the present invention, the compound may be administered in combination with various therapeutic agents or preventive agents for diseases that are considered to exhibit the efficacy thereof. The combination may be administered simultaneously, separately, concurrently, and continuously or at desired time intervals. The co-administered agents may be blended or formulated separately. The therapeutic agent may be, for example, a farnesyl transferase inhibitor such as tetrapeptides having or mimicking the CAAX motif or analogs of farnesyl pyrophosphate, e.g., lonafarnib or tipifarnib (ZARNESTRA®) (see U.S. Pat. No. 8,828,356). The therapeutic agent may be, for example, a statin (e.g., pravastatin, lovastatin, simvastatin, fluvastatin, cerivastatin, atorvastatin, mevastatin, pitavastatin) and/or an amino-bisphosphonate (e.g., zoledronic acid (zoledronate), risedronic acid (risedronate), ibandronic acid (ibandronate), alendronic acid (alendronate), olpandronic acid (olpandronate), neridronic acid (neridronic acid), pamidronic acid (pamidronate)) and/or an mTOR inhibitor (e.g., rapamycin (or sirolimus), everolimus, temsirolimus, deforolimus, ridaforolimus, nab-rapamycin, salirasib) (see U.S. Pat. Nos. 9,381,203; 10,098,871). The therapeutic agent may be, for example, a progerin expression inhibitor, such as pVHL (Hippel-Lindau tumor suppressor protein)-progerin binding promoter, or an RNA molecular inhibitory of progerin expression, such as an antisense-RNA, interference RNA, short-hairpin RNA, and small interfering RNA (siRNA) (see U.S. Pat. Nos. 9,249,153; 9,326,992; 9,833,468). The therapeutic agent may be, for example, an agent that inhibits binding of a SIRT6 expression inhibitor to a 3′UTR region of human mRNA encoding SIRT6 (see U.S. Pat. Nos. 9,650,637 and 10,036,023).

The methods of the present invention find use in both veterinary and medical applications. Suitable subjects include avians, reptiles, amphibians, fish, and mammals. The term “mammal” as used herein includes, but is not limited to, humans, primates, non-human primates (e.g., monkeys and baboons), cattle, sheep, goats, pigs, horses, cats, dogs, rabbits, rodents (e.g., rats, mice, hamsters, and the like), etc. Human subjects include neonates, infants, juveniles, and adults. Optionally, the subject is “in need of” the methods of the present invention, e.g., because the subject has or is believed at risk for a disorder including those described herein or that would benefit from the delivery of a compound as described herein. As a further option, the subject can be a laboratory animal and/or an animal model of disease. Preferably, the subject is a human.

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A method of treating, delaying the onset of, slowing the progression of, or reducing the severity of a premature aging disorder in a subject in need thereof, comprising administering to the subject a therapeutically effective amount of the compound of Formula 1 or a pharmaceutically acceptable salt thereof: 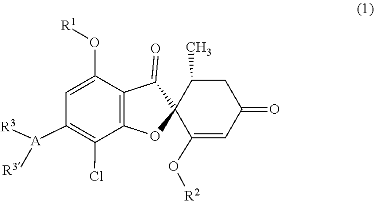
wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH═, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
Substituent group Y is a C1-C6 alkyl group, a C1-C6 alkoxy group, a halogen atom, or a hydroxy group,
thereby treating, delaying the onset of, slowing the progression of, or reducing the severity of the premature aging disorder.

2. The method of claim 1, wherein the premature aging disorder is Hutchinson—Gilford progeria syndrome, Werner syndrome, Bloom syndrome, Rothmund—Thomson syndrome, Cockayne syndrome, xeroderma pigmentosum, trichothiodystrophy, combined xeroderma pigmentosum-Cockayne syndrome, or restrictive dermopathy.

3. (canceled)

4. A method of treating, delaying the onset of, slowing the progression of, or reducing the severity of an age-related disease in a subject in need thereof, comprising administering to the subject a therapeutically effective amount of a compound of Formula 1 or a pharmaceutically acceptable salt thereof: 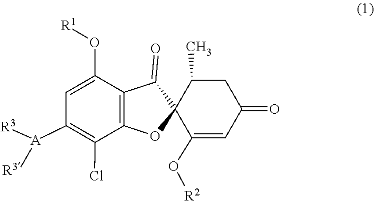
wherein:
R¹ is a C1-C6 alkyl group optionally substituted with the same or different one to two substituents selected from a substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X,
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, —CH═, or a cyano group, wherein when A is a cyano group, R³ and R^{3′} do not exist,
R³ and R^{3′} are each independently a hydrogen atom, a halogen atom, a cyano group, a hydroxy group, an oxo group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X, or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from a substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group,
Substituent group Y is a C1-C6 alkyl group, a C1-C6 alkoxy group, a halogen atom, or a hydroxy group,
thereby treating, delaying the onset of, slowing the progression of, or reducing the severity of the age-related disease.

5. The method of claim 4, wherein the age-related disease is type 2 diabetes, neurodegenerative disease, cancer, cardiovascular disease, obesity, increased cholesterol levels, hypertension, ocular disorders, cataracts, glaucoma, osteoporosis, blood clotting disorders, arthritis, hearing loss, stroke, Alzheimer's disease, osteoarthritis, rheumatoid arthritis, chronic obstructive pulmonary disease, fatty liver disease, or nonalcoholic steatohepatitis.

6. The method of claim 1, wherein the 5-membered aromatic heterocyclic ring or the 5-membered aromatic heterocyclic group in A, R³, or R³ is any one selected from the group:

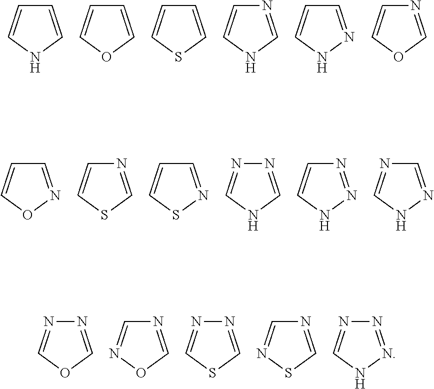

7. The method of claim 1, wherein the 6-membered aromatic heterocyclic ring or the 6-membered aromatic heterocyclic group in A, R³, or R^{3′} is any one selected from the group:

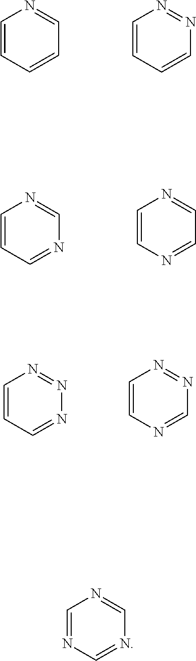

8. The method of claim 1, wherein the 8-10 membered condensed aromatic heterocyclic ring or the 8-10 membered condensed aromatic heterocyclic group in A, R³, or R^{3′} is any one selected from the group:

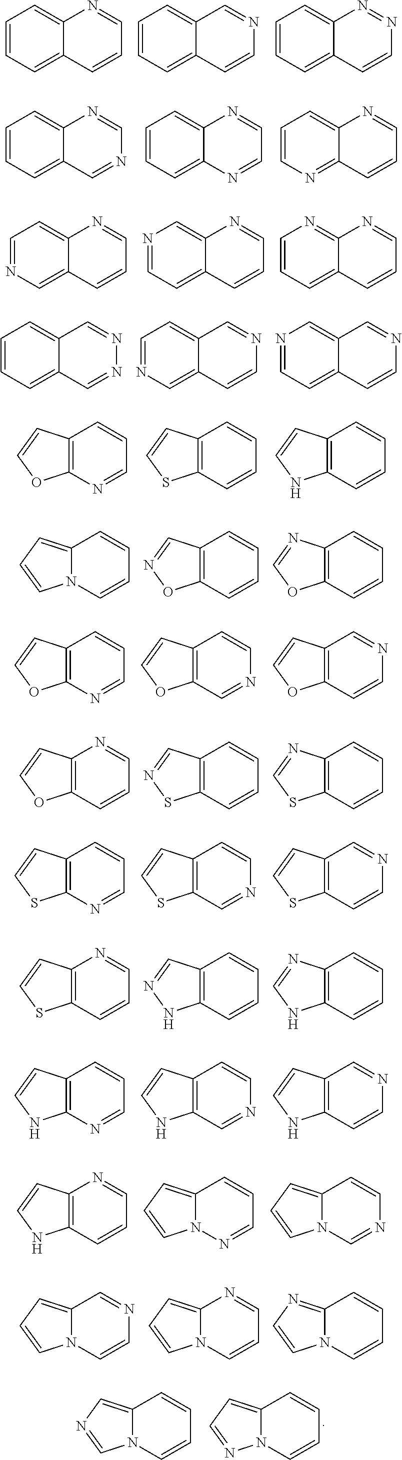

9. The method of claim 1, wherein the 5-7 membered unsaturated heterocyclic ring or 5-7 membered unsaturated heterocyclic group in A, R³, or R^{3′} is any one selected from the group:

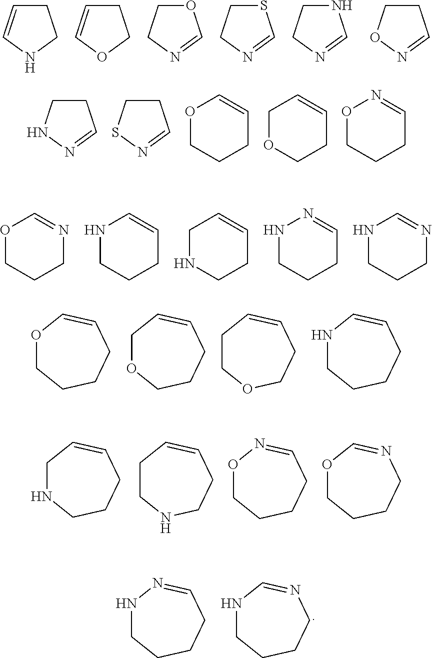

10. The method of claim 1, wherein the 4-7 membered saturated heterocyclic ring or the 4-7 membered saturated heterocyclic group in A, R¹, R², or R³ is any one selected from the group:

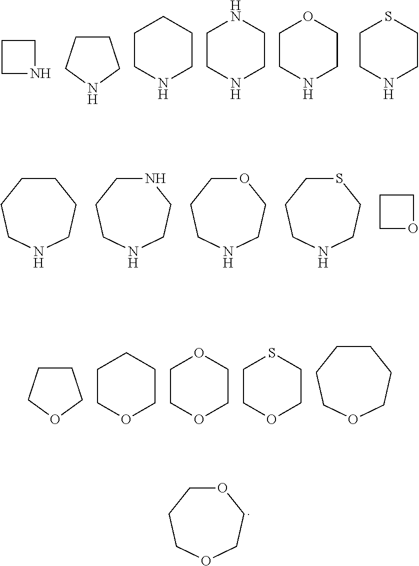

11. The method of claim 1, wherein the compound of Formula 1 is any compound selected from the following group:
(2S,5′R)-7-chloro-6-(5-ethyl-1,3,4-oxadiazol-2-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro [benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl] spiro [benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro [benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro [benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(1H-pyrazol-5-yl) spiro [benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro [benzofuran-2,4′-cyclohex-2-ene]-1′,3-dione;
(2S,5′R)-7-chloro-6-(1,8-dioxa-2-azaspiro[4.5] dec-2-en-3-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5] dec-2-en-3-yl) spiro [benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro [benzofuran-2,4′-cyclohex-2-ene]-1′,3-dione;
(2S,5′R)-7-chloro-3′,4-dimethoxy-6-(6-methoxy-3-pyridyl)-5′-methyl-spiro [benzofuran-2,4′-cyclohex-2-ene]-1′,3-dione; or
(2S,5′R)-7-chloro-3′,4-dimethoxy-5′-methyl-6-(3-pyridyl) spiro [benzofuran-2,4′-cyclohex-2-ene]-1′,3-dione.

12. The method of claim 1, wherein the compound of Formula 1 is a compound of Formula 1′ or a pharmacologically acceptable salt thereof: 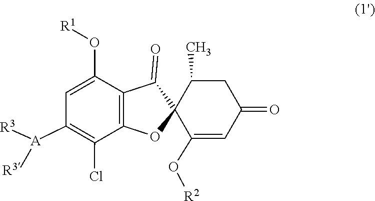
wherein:
R¹ is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X
R² is a C1-C6 alkyl group optionally substituted with the same or different one to two substituents selected from the substituent group X,
a C3-C6 cycloalkyl group optionally substituted with the same or different one to two substituents selected from the substituent group X, or
a 4-7 membered saturated heterocyclic group optionally substituted with the same or different one to two substituents selected from the substituent group X.
A is a 5-membered aromatic heterocyclic ring,
a 6-membered aromatic heterocyclic ring,
an 8-10 membered condensed aromatic heterocyclic ring,
a 5-7 membered unsaturated heterocyclic ring,
a 4-7 membered saturated heterocyclic ring,
a benzene ring, or a single bond, wherein when it is a single bond, one or the other of R³ and R^{3′} is not present,
R³ and R^{3′} are each independently a hydrogen atom, a halogen atom, a cyano group, a hydroxy group,
a C1-C6 alkyl group optionally substituted with the same or different one to two substituents selected from the substituent group X,
a C1-C6 alkoxy group optionally substituted with the same or different one to two substituents selected from the substituent group X,
a C2-C6 alkenyl group optionally substituted with the same or different one to two substituents selected from the substituent group X,
a C2-C6 alkynyl group optionally substituted with the same or different one to two substituents selected from the substituent group X,
a C3-C6 cycloalkyl group optionally substituted with the same or different one to two substituents selected from the substituent group X,
an amino group optionally substituted with the same or different one to two substituents selected from the substituent group X,
a C1-C6 alkoxycarbonyl group optionally substituted with the same or different one to two substituents selected from the substituent group X,
a carbamoyl group optionally substituted with the same or different one to two substituents selected from the substituent group X,
a phenyl group optionally substituted with the same or different one to two substituents selected from the substituent group X,
a 5-membered aromatic heterocyclic group optionally substituted with the same or different one to two substituents selected from the substituent group X,
a 6-membered aromatic heterocyclic group optionally substituted with the same or different one to two substituents selected from the substituent group X,
a 5-7 membered unsaturated heterocyclic group optionally substituted with the same or different one to two substituents selected from the substituent group X,
a 4-7 membered saturated heterocyclic group optionally substituted with the same or different one to two substituents selected from the substituent group X,
an 8-10 membered condensed aromatic heterocyclic group optionally substituted with the same or different one to two substituents selected from the substituent group X,
or
R³ and R^{3′} may form a 5-7 membered unsaturated heterocyclic ring, a 4-7 membered saturated heterocyclic ring, or a C3-C6 cycloalkyl ring as a ring that binds to each other and condenses with A, and the ring is optionally substituted with the same or different one to two substituents selected from the substituent group X,
Substituent group X is a halogen atom, a cyano group, a hydroxy group, an oxo group, a C1-C6 alkyl group, a hydroxy C1-C6 alkyl group, a C1-C6 alkoxy C1-C6 alkyl group, a C1-C6 haloalkyl group, a C3-C6 cycloalkyl group, a C3-C6 halocycloalkyl group,
a phenyl group optionally substituted with the same or different one to two substituents selected from the substituent group Y,
a 5-membered aromatic heterocyclic group optionally substituted with the same or different one to two substituents selected from the substituent group Y,
a 6-membered aromatic heterocyclic group optionally substituted with the same or different one to two substituents selected from the substituent group Y,
a 4-7 membered saturated heterocyclic group optionally substituted with the same or different one to two substituents selected from the substituent group Y,
a C1-C6 alkoxy group, a C1-C6 haloalkoxy group, a C3-C6 cycloalkoxy group, a C3-C6 halocycloalkoxy group,
a phenoxy group optionally substituted with the same or different one to two substituents selected from the substituent group Y,
a 5-membered aromatic heterocyclic oxy group optionally substituted with the same or different one to two substituents selected from the substituent group Y,
a 6-membered aromatic heterocyclic oxy group optionally substituted with the same or different one to two substituents selected from the substituent group Y,
a 4-7 membered saturated heterocyclic oxy group optionally substituted with the same or different one to two substituents selected from the substituent group Y,
a C1-C6 alkoxycarbonyl group, a C3-C6 cycloalkoxycarbonyl group, a carboxy group, a C1-C6 alkylcarbonyl group, a C3-C6 cycloalkylcarbonyl group,
a phenylcarbonyl group optionally substituted with the same or different one to two substituents selected from the substituent group Y,
a carbamoyl group, a mono (C1-C6 alkyl) aminocarbonyl group, a di (C1-C6 alkyl) aminocarbonyl group, a mono (C1-C6 alkyl) aminosulfonyl group, a di (C1-C6 alkyl) aminosulfonyl group, an amino group, a mono (C1-C6 alkyl) amino group, a di (C1-C6 alkyl) amino group, a C1-C6 alkoxycarbonylamino group, a mono (C1-C6 alkyl) aminocarbonylamino group, a di (C1-C6 alkyl) aminocarbonylamino group, a C1-C6 alkylcarbonylamino group,
a phenylcarbonylamino group optionally substituted with the same or different one to two substituents selected from the substituent group Y,
a 5-membered aromatic heterocyclic carbonylamino group optionally substituted with the same or different one to two substituents selected from the substituent group Y,
a 6-membered aromatic heterocyclic carbonylamino group optionally substituted with the same or different one to two substituents selected from the substituent group Y, or
a C1-C6 alkylsulfonylamino group, and
Substituent group Y is a C1-C6 alkyl group, a C1-C6 alkoxy group, a halogen atom, or a hydroxy group.

13. The method of claim 12, wherein R¹ is a methyl group, an ethyl group, or a hydroxyethyl group.

14. The method of claim 12, wherein R² is a methyl group.

15. The method of claim 12, wherein the 5-membered aromatic heterocyclic ring or the 5-membered aromatic heterocyclic group in A, R³, or R^{3′} is any one selected from the group:

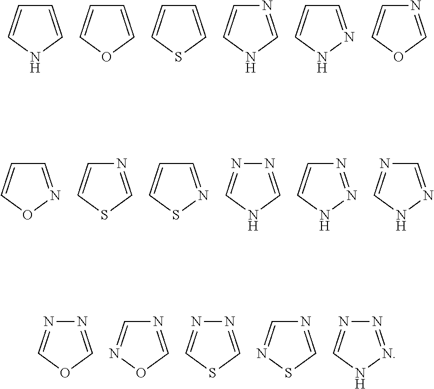

16. The method of claim 12, wherein the 6-membered aromatic heterocyclic ring or the 6-membered aromatic heterocyclic group in A, R³, or R^{3′} is any one selected from the group:

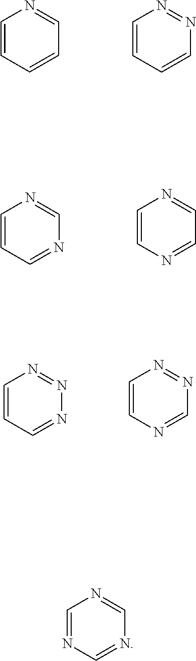

17. The method of claim 12, wherein the 5-7 membered unsaturated heterocyclic ring or the 5-7 membered unsaturated heterocyclic group in A, R³, or R^{3′} is any one selected from the group:

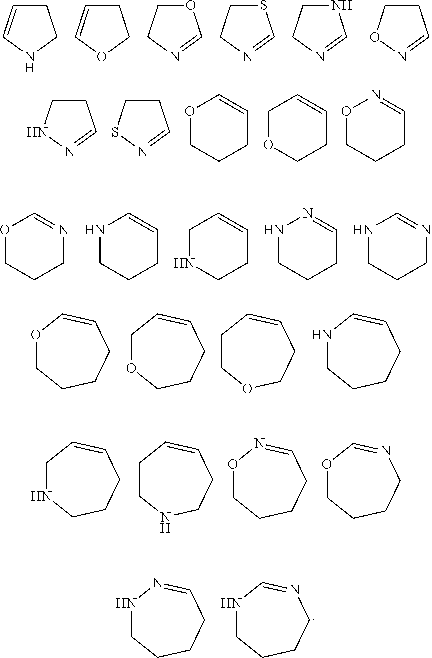

18. The method of claim 12, wherein the 4-7 membered saturated heterocyclic ring or the 4-7 membered saturated heterocyclic group in A, R¹, R², or R³ is any one selected from the group:

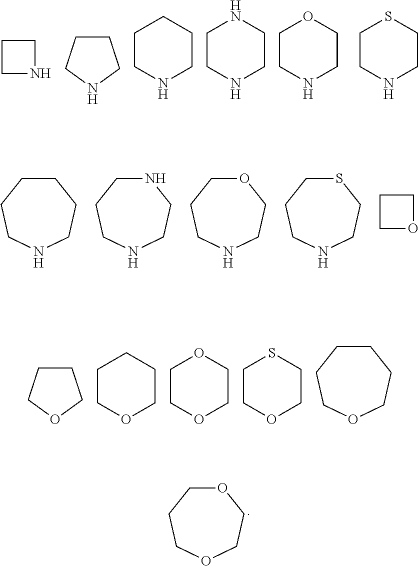

19. The method of claim 12, wherein A is a 5-membered aromatic heterocyclic ring, R³ is a methyl group, an ethyl group, a hydroxy C1-C3 alkyl group, or a methoxy C1-C3 alkyl group, and R^{3′} is a hydrogen atom.

20. The method of claim 12, wherein A is any ring selected from the following group, and in the case of two binding groups, R^{3′} is not present: 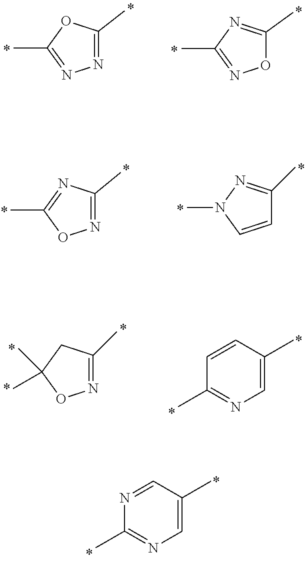
wherein * indicates a binding group.

21. The method of claim 12, wherein the compound of Formula 1 is a compound of a Formula 1″ or a pharmacologically acceptable salt thereof: 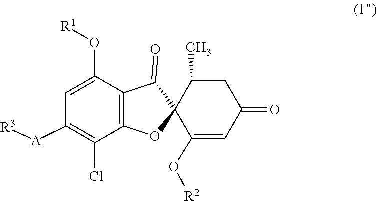
wherein R¹ is a methyl group or an ethyl group;
R² is a methyl group;
A is any ring selected from the following group: 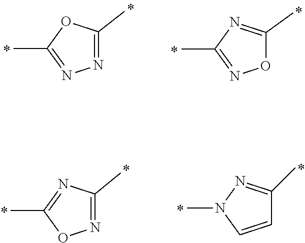
* indicates a binding group; and
R³ is a methyl group or an ethyl group.

22. The method of claim 12, wherein the compound of Formula 1′ is any compound selected from the following group:
(2S,5′R)-7-chloro-6-(2-hydroxyethoxy)-3′,4-dimethoxy-5′-methyl-spiro [benzofuran-2,4′-cyclohex-2-ene]-1′,3-dione;
(2S,5′R)-7-chloro-3′,4-dimethoxy-6-(2-methoxyethoxy)-5′-methyl-spiro [benzofuran-2,4′-cyclohex-2-ene]-1′,3-dione;
(2S,5′R)-7-chloro-3′,4-dimethoxy-5′-methyl-6-(1-methylpyrazol-3-yl) spiro [benzofuran-2,4′-cyclohex-2-ene]-1′,3-dione;
(2S,5′R)-7-chloro-6-(1-ethylpyrazol-3-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(5-methyl-1,3,4-oxadiazol-2-yl) spiro [benzofuran-2,4′-cyclohex-2-ene]-1′,3-dione;
(2S,5′R)-7-chloro-3′,4-dimethoxy-5′-methyl-6-(3-methyl-1,2,4-oxadiazol-5-yl) spiro [benzofuran-2,4′-cyclohex-2-ene]-1′,3-dione;
(2S,5′R)-7-chloro-3′,4-dimethoxy-5′-methyl-6-(5-methyl)-1,2,4-oxadiazol-3-yl) spiro [benzofuran-2,4′-cyclohex-2-ene]-1′,3-dione;
(2S,5′R)-7-chloro-6-[5-(1-hydroxy-1-methyl-ethyl)-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-6-[5-[(1S)-1-hydroxyethyl]-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-6-[5-[(1R)-1-hydroxyethyl]-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-4-ethoxy-6-[5-(1-hydroxy-1-methyl-ethyl)-1,3,4-oxadiazol-2-yl]-3′-methoxy-5′-methyl-spiro [benzofuran-2,4′-cyclohex-2-ene]-1′,3-dione;
(2S,5′R)-7-chloro-4-ethoxy-6-[5-[(1S)-1-hydroxyethyl]-1,3,4-oxadiazol-2-yl]-3 ‘-methoxy-5’-methyl-spiro [benzofuran-2,4′-cyclohex-2-ene]-1′,3-dione;
(2S,5′R)-7-chloro-6-[3-(1-hydroxyethyl)-1,2,4-oxadiazol-5-yl]-3′,4-dimethoxy-5′-methyl-spiro [benzofuran-2,4′-cyclohex-2-ene]-1′,3-dione;
(2S,5′R)-7-chloro-4-(2-hydroxyethoxy)-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-4-(2-hydroxyethoxy)-3′-methoxy-5′-methyl-6-(3-methyl-1,2,4-oxadiazol-5-yl) spiro [benzofuran-2,4′-cyclohex-2-ene]-1′,3-dione;
(2S,5′R)-7-chloro-4-(2-hydroxyethoxy)-3′-methoxy-5′-methyl-6-(5-methyl-1,2,4-oxadiazol-3-yl) spiro [benzofuran-2,4′-cyclohex-2-ene]-1′,3-dione;
(2S,5′R)-7-chloro-6-(5-ethyl-1,3,4-oxadiazol-2-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(5-tetrahydropyran-4-yl-1,3,4-oxadiazol-2-yl) spiro [benzofuran-2,4′-cyclohex-2-ene]-1′,3-dione;
(2S,5′R)-7-chloro-3′,4-dimethoxy-5′-methyl-6-[5-(1-methyl-4-piperidyl)-1,3,4-oxadiazol-2-yl] spiro [benzofuran-2,4′-cyclohex-2-ene]-1′,3-dione;
(2S,5′R)-7-chloro-6-[5-(4-fluoro-1-methyl-4-piperidyl)-1,3,4-oxadiazol-2-yl]-3′,4-dimethoxy-5′-methyl-spiro [benzofuran-2,4′-cyclohex-2-ene]-1′,3-dione;
(2S,5′R)-7-chloro-3′,4-dimethoxy-6-[5-[(1S)-1-methoxyethyl]-1,3,4-oxadiazol-2-yl]-5′-methyl-spiro [benzofuran-2,4′-cyclohex-2-ene]-1′,3-dione;
(2S,5′R)-7-chloro-4-ethoxy-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-4-(difluoromethoxy)-3′-methoxy-5′-methyl-6-(5-methyl-1,3,4-oxadiazol-2-yl) spiro [benzofuran-2,4′-cyclohex-2-ene]-1′,3-dione;
(2S,5′R)-7-chloro-3′,4-dimethoxy-6-[3-(1-methoxyethyl)-1,2,4-oxadiazol-5-yl]-5′-methyl-spiro [benzofuran-2,4′-cyclohex-2-ene]-1′,3-dione;
(2S,5′R)-7-chloro-6-[3-(1-hydroxy-1-methyl-ethyl)-1,2,4-oxadiazol-5-yl]-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(1H-pyrazol-5-yl) spiro [benzofuran-2,4′-cyclohex-2-ene]-1′,3-dione;
(2S,5′R)-7-chloro-3′,4-dimethoxy-6-[1-(2-methoxyethyl) pyrazol-3-yl]-5′-methyl-spiro [benzofuran-2,4′-cyclohex-2-ene]-1′,3-dione;
(2S,5′R)-7-chloro-6-(1,8-dioxa-2-azaspiro [4.5] dec-2-en-3-yl)-3′,4-dimethoxy-5′-methyl-spiro [benzofuran-2,4′-cyclohex-2-ene]-1′,3-dione;
(2S,5′R)-7-chloro-3′,4-dimethoxy-5′-methyl-6-(8-methyl-1-oxa-2,8-diazaspiro[4.5] dec-2-en-3-yl) spiro [benzofuran-2,4′-cyclohex-2-ene]-1′,3-dione;
(2S,5′R)-7-chloro-3′,4-dimethoxy-6-(2-methoxypyrimidin-5-yl)-5′-methyl-spiro [benzofuran-2,4′-cyclohex-2-ene]-1′,3-dione;
(2S,5′R)-7-chloro-3′,4-dimethoxy-6-(6-methoxy-3-pyridyl)-5′-methyl-spiro [benzofuran-2,4′-cyclohex-2-ene]-1′,3-dione;
(2S,5′R)-7-chloro-3′,4-dimethoxy-5′-methyl-6-(3-pyridyl) spiro [benzofuran-2,4′-cyclohex-2-ene]-1′,3-dione; or
(2S,5′R)-7-chloro-3′,4,6-trimethoxy-5′-methyl-spiro [benzofuran-2,4′-cyclohex-2-ene]-1′,3-dione.

23-27. (canceled)
